(19) United States
(12) Patent Application Publication
(10) Pub. No.: US 2014/0148792 A1
(43) Pub. Date: May 29, 2014

(54) Laser-Activated Drug Delivery Device

(71) Applicant: On Demand Therapeutics Inc., Menlo Park, CA (US)

(72) Inventors: Jonathan R. Coppeta, Windham, NH (US); Robert Dyer, Concord, MA (US); Vipul Taneja, North Chelmsford, MA (US); John T. Santini, JR., Chanhassen, MN (US)

(73) Assignee: On Demand Therapeutics Inc., Menlo Park, CA (US)

(21) Appl. No.: 14/170,994

(22) Filed: Feb. 3, 2014

Related U.S. Application Data
(63) Continuation of application No. PCT/US2012/050240, filed on Aug. 10, 2012.
(60) Provisional application No. 61/522,219, filed on Aug. 10, 2011.

(51) Int. Cl. A61F 9/00

(52) U.S. Cl. CPC A61F 9/0017
USPC 604/521; 604/93.01

(57) ABSTRACT

Devices and methods are provided for selectively delivering a drug by laser activation. The device includes structural elements forming a hermetic, enclosed reservoir, and at least one drug unit contained in the enclosed reservoir. The at least one drug unit includes a drug. The device is able to absorb laser irradiation effective to open the enclosed reservoir to permit release of the drug. The method includes implanting a drug delivery device into a tissue site of a patient, and irradiating at least a portion of the drug delivery device to breach the enclosed reservoir to permit the drug to be released in tissues at the tissue site.

### CROSS-REFERENCE TO RELATED APPLICATIONS

This application is a continuation of International Application No. PCT/US2012/050240, filed Aug. 10, 2012, which claims priority benefit of U.S. Provisional Patent Application No. 61/522,219 filed Aug. 10, 2011. These applications are incorporated herein by reference.

### BACKGROUND

The present disclosure is directed generally to improved, implantable drug delivery devices and, more particularly, to laser-activated drug delivery devices, which may, for example, be used to administer a drug to the eye of a patient.

Drug-eluting devices that may be implanted into the eye are known. These devices may be surgically implanted or are injected into the posterior chamber or into, onto, or under layers of the eye such as the conjunctiva, sclera, or choroid. Commercially available drug eluting implants include ganciclovir implants (Vitrasert®) for treatment of CMV retinitis in patients with acquired immunodeficiency syndrome (AIDS), fluocinolone acetonide implants (e.g., Retisert®) for treatment of chronic non-infectious uveitis of the posterior segment of the eye, and dexamethasone implants (e.g., Ozurdex®) for treatment of macular edema caused by retinal vein occlusions and for chronic non-infectious uveitis of the posterior segment of the eye. Notable implants in development include an injectable fluocinolone acetonide implant (Illuvien™) for the treatment of diabetic macular edema (DME) and a bioerodible latanoprost implant (Durasert™) for treatment of glaucoma and ocular hypertension. Typically, such implants release a drug at a constant or slowly changing rate. See, U.S. Pat. Nos. 6,217,895 and 6,548,078 (to Retisert), U.S. Pat. No. 5,378,475 (to Vitrasert), U.S. Pat. Nos. 6,726,918; 6,899,717; 7,033,605; 7,625,582; 7,767,223 (to Ozurdex), and U.S. Patent Application Publication 2007/0122483 (to Iluvien). These implantable devices typically provide a constant pharmacokinetic profile resulting from a continuous drug dosing. This continuous dosing may be acceptable for certain drugs, but for other drugs, continuous dosing can result in serious side effects. For example, continuous delivery of a steroid in the eye results in a high incidence of cataracts or elevated intraocular pressure that may result in glaucoma. Thus, in some cases, it is desirable to deliver the drug only when needed, for example at spaced time intervals.

Another significant challenge in the development of technologies for the delivery of pharmaceutical drugs and, in particular, macromolecule (e.g., peptide and protein) drugs, is the limited stability of these molecules when in contact with water vapor or when in an aqueous solution. Many macromolecule drugs, including proteins, that are unstable in aqueous solution are handled and stored as dry solids (“dry” is defined herein means substantially free of residual moisture, typically with a water content not exceeding 10% water by weight). Delivery systems that store or release macromolecule drugs in liquid or gel form will have limited utility due to accelerated degradation of the drug caused by high residual moisture. If a macromolecule drug can be kept in a dry, solid form, then its degradation can be minimized and a long-term implantable device is possible. See, Proos, et al., “Long-term Stability and In Vitro Release of hPTH(1-34) from a Multi-reservoir Array” Pharmaceutical Research, Vol. 25, No. 6, Pages 1387-95 (2008). It is therefore desirable to create a drug delivery system that has the ability to store a drug in a dry, solid form and that prohibits or limits any moisture from passing through the device and into the drug, until such time that release of the drug is desired.

PCT WO 2009/097468 to Kliman discloses drug delivery devices that may be configured for implantation into an ocular region of a subject that may be triggered by an optical stimulus, such as light having a certain wavelength.

There remains a need, however, for an improved implantable drug delivery device for delivering a drug to the interior of the eye. In particular, a need exists for an implantable drug delivery device having a simple and compact construction that provides a hermetic barrier to protect a sensitive drug payload until the payload is selectively released, and that is capable of laser activation so that a drug dosing can be initiated at a selected time using non-invasive techniques. Desirably, such a device should be easy to manufacture and should not require on-board electronics.

### SUMMARY

In one aspect, devices are provided for containing and releasing a drug. One embodiment includes an implantable drug delivery device that uses a tube element and multiple end cap elements to contain and release at least one drug. The device includes a tube element having a first open end and a second open end. The device also includes a first end cap joined to the first open end of the tube element at a first joint, and a second end cap element joined to the second end of the tube element at a second joint. The tube element, the first end cap element, and the second end cap element form an enclosed reservoir in which at least one drug unit is contained. The at least one drug unit includes a drug. The device is able to absorb light irradiation from a laser source effective to open the enclosed reservoir to permit release of the drug.

The first end cap element and the second end cap element may be joined to the tube element by welding or soldering at the first joint and the second joint. In some cases, the first end cap element and the second end cap element are joined to the tube element by adhesive at the first joint and the second join, and the device may further include a coating over the adhesive at the first joint and the second joint, forming a hermetic seal over the first joint and the second joint. The tube element itself may be able to absorb the light irradiation, or in other cases, the device further includes a coating over the tube element, wherein the coating is able to absorb the light irradiation. In one embodiment, the device further includes multiple barrier elements positioned within the enclosed reservoir, which barrier elements define a plurality of separate reservoir sections. Drug units may be distributed within the separate reservoir sections.

In another embodiment, the implantable drug delivery device includes multiple cup elements and a coating, which together form the sealed reservoir(s) containing at least one drug. The device may include a first cup element having an open end and a closed end, and a second cup element having an open end and a closed end. The device may further include a coating over all or a portion of the first cup element and the second cup element, and the coating may form a hermetic seal between the first cup element and the second cup element. The first cup element, the second cup element, and the coating form an enclosed reservoir in which at least one drug unit is contained. The at least one drug unit includes a drug. The device is configured to absorb light irradiation from a laser source effective to open the enclosed reservoir to permit release of the drug.

The first cup element and the second cup element may be spaced apart from one another to define a gap, and the coating fills the gap. In one case, the open end of the first cup overlaps the open end of the second cup. The coating may be one that is capable of absorbing the light irradiation, and/or the first cup element or the second cup element is able to absorb the light irradiation.

In another aspect, methods are provided for delivering a drug to a patient using the presently disclosed devices. The methods include implanting one of the drug delivery devices at or into a tissue site of the patient, and then irradiating at least a portion of the drug delivery device to breach the enclosed reservoir to permit the drug to be released at the tissue site.

### BRIEF DESCRIPTION OF THE DRAWINGS

FIG. 1A depicts a perspective view of an implantable drug delivery device according to one embodiment, with a partial cutaway of a coating and a tube element to show the underlying construction of the device.

FIG. 1B depicts an exploded, perspective view of the tube element, a drug unit, and end cap elements of the implantable drug delivery device of FIG. 1A.

FIG. 1C depicts a perspective view of an implantable drug delivery device according to one embodiment, with a partial cutaway of a coating and a tube element to show the underlying construction of the device.

FIG. 1D depicts a perspective view of an implantable drug delivery device according to one embodiment, with a partial cutaway of a coating and tube elements to show the underlying construction of the device.

FIG. 2A depicts a perspective view of an implantable drug delivery device according to one embodiment, with a partial cutaway of a coating, cup elements, and a tube element to show the underlying construction of the device.

FIG. 2B depicts an exploded, perspective view of the cup elements, a drug unit, and the tube element of the implantable drug delivery device of FIG. 2A.

FIG. 3A depicts a perspective view of an implantable drug delivery device according to one embodiment, with a partial cutaway of a coating and cup elements to show the underlying construction of the device.

FIG. 3B depicts an exploded, perspective view of the cup elements and a drug unit of the implantable drug delivery device of FIG. 3A.

FIG. 4A depicts a perspective view of an implantable drug delivery device according to one embodiment.

FIG. 4B depicts an exploded, perspective view of cup elements and drug units of the implantable drug delivery device of FIG. 4A.

FIG. 4C depicts a perspective view of an implantable drug delivery device according to one embodiment.

FIG. 4D depicts an exploded, perspective view of cup elements, drug units, and an end cap element of the implantable drug delivery device of FIG. 4C.

FIG. 5 depicts a graph comparing the sealing risk and the water vapor transmission rate of the implantable drug delivery devices of FIGS. 1A, 2A, and 3A.

### DETAILED DESCRIPTION

The embodiments described herein relate to implantable drug delivery devices (DDDs) that provide one or more hermetically-sealed reservoirs and that are capable of providing laser-activated release of a drug. In particular, the DDDs are capable of being implanted into a tissue of the eye and subsequently activated by an ocular laser to permit the drug to be released in ocular tissues. Because the reservoirs are hermetically sealed, they advantageously are highly impermeable to water, water vapor, or reactive gases such as oxygen. Thus, the devices are capable of providing long-term viability for drugs that are sensitive to chemical change, such as, for example, degradation by moisture or oxygen.

The devices and methods described herein allow for the selective release of a drug by laser irradiation of a drug delivery device having a hermetically-sealed reservoir or reservoirs. This is especially useful for devices that have been implanted in the transparent tissues of the eye, in which the non-invasive and efficacious introduction of a laser beam may be easily accomplished. The devices described herein are formed of materials and arranged in structures that provide the drug in a hermetic reservoir capable of being breached by laser irradiation.

Certain constraints exist in forming an implantable DDD having a hermetically-sealed reservoir for containing and releasing a drug. For example, to facilitate implantation, the DDD should have a dimension sufficiently small so as to allow injection into or implantation at an ocular tissue site. The DDD also should be sufficiently rigid to withstand implantation while maintaining the hermetic seal over the reservoir. Additionally, structural joints of the DDD should be robustly formed to prevent compromise of the reservoir during implantation and while the DDD is implanted in the tissue prior to activation. Furthermore, the DDD should have a simple construction that is easy to manufacture from biocompatible materials and assemble for use.

Other constraints exist in forming an implantable DDD that is capable of providing a laser-activated release of a drug. For example, at least a portion of the DDD should be able to absorb light irradiation from a laser source effective to open the hermetically-sealed reservoir to permit the drug to be released. The irradiation-absorbing portion should be large enough to be specifically targeted by an ocular laser. Additionally, the irradiation-absorbing portion should be formed of a biocompatible material that is capable of being breached upon exposure to a minimal amount of laser energy. Furthermore, the irradiation-absorbing portion should have a thickness that is capable of being breached upon exposure to a minimal amount of laser energy.

The implantable DDDs described herein have been developed to provide a desirable balance in addressing the competing constraints associated with a DDD that has a hermetically-sealed reservoir and that provides a laser-activated release of a drug. The elements of the DDD are relatively easy to manufacture from biocompatible metals and glasses, and assembly is relatively straightforward due to the simple features of construction.

For example, according to one embodiment, the implantable DDD includes a glass tube element, metal end cap elements, and a metal coating, which together provide an appropriate reservoir for containing and releasing a drug. The reservoir is formed by the glass tube element and the metal end cap elements joined to opposing ends of the glass tube element. The metal end cap elements may be joined to the glass tube element by an adhesive, ensuring that the metal end cap elements are securely bonded to the glass tube. The metal coating forms a seal over the joints, ensuring that the reservoir is hermetically sealed to maintain the integrity of the drug. The metal coating also may cover some or all of the glass tube element to provide a target area capable of absorbing light irradiation from a laser source effective to open the hermetically-sealed reservoir to permit the drug to be released. In embodiments, the metal coating and the glass tube element are sufficiently rigid to withstand implantation, yet they are able to be breached upon exposure to a minimal amount of laser energy.

According to another embodiment, the implantable DDD includes a metal tube element and metal end cap elements, which together provide an appropriate reservoir for containing and releasing a drug. The reservoir is formed by the metal tube element and metal end cap elements joined to opposing ends of the metal tube element. The metal end cap elements may be joined to the metal tube element by welding or soldering, ensuring that the reservoir is hermetically sealed to maintain the integrity of the drug. In embodiments, the metal tube element is able to absorb light irradiation from a laser source effective to open the hermetically-sealed reservoir to permit the drug to be released. The metal tube element is sufficiently rigid to withstand implantation, yet it is able to be breached upon exposure to a minimal amount of laser energy.

In variations of the foregoing embodiments, the glass and metal materials used for the tube element and end caps may be substituted with other materials of constructions, such as other ceramics or silicon, providing the necessary reservoir structure and hermeticity.

The hermetically-sealed reservoirs of the implantable DDDs described herein beneficially enable the use of sensitive drugs in an implant device intended for deployment in a patient over an extended period. For example, some treatment regimens require sustained or multiple releases of a drug over a period ranging from a week to several months, a year, or more. For drugs that are sensitive to water or air exposure, a hermetically-sealed reservoir protects the drug payload and eliminates or minimizes drug degradation over the extended period.

In embodiments, the implantable DDDs described herein are laser-activated and thus facilitate non-invasive release of a drug to the ocular tissue being treated, such as the macula or retina, by releasing the drug into the posterior chamber through the vitreous portion of the eye or through the conjunctiva, sclera, or choroid. In addition to being non-invasive, laser activation allows for multiple dosing from a single implanted drug delivery device. Multiple dosing allows the dosing interval to be tailored, providing some control over the drug concentration over time. Laser activation also allows a physician to precisely control the initiation of treatment and administer arbitrary and customized treatment regimens. The selectable nature of the activation and dosing is not realized in existing passive drug delivery device implants.

The individual reservoirs or reservoir sections of the implantable DDDs described herein are independent of the drug formulation and allow the integration of different drug forms and types in the overall device. By encapsulating a different drug in each reservoir or reservoir section, an optimal formulation for each drug can be developed. The overall device therefore can enable multiple drug therapies within one implantable device. In addition, when appropriate, multiple drugs can be co-formulated within one reservoir or reservoir section.

### 1. Laser-Activated Drug Delivery Devices

The embodiments described herein include DDDs that can be implanted into an ocular tissue with minimal intervention, are hermetically sealed to protect a drug payload over time, and can be laser activated to selectively release the drug as needed. In embodiments, the primary components of the DDD embodiments described herein include: structural elements forming an enclosed reservoir, and at least one drug unit contained in the enclosed reservoir. The drug unit includes at least one drug. The enclosed reservoir is hermetically sealed by the structural elements and/or a coating to maintain the biologic activity or chemical viability of the drug until the reservoir is intentionally breached (by application of laser energy) to permit the drug to be released to one or more target tissues at or around the site of implantation. For example, in some embodiments, one or more of the structural elements are formed of a hermetic material, preventing air and water from entering the enclosed reservoir. In some embodiments, the coating may form a hermetic seal over joints of the structural elements to prevent air and water from entering the enclosed reservoir. In some embodiments, one or more of the structural elements is formed of a non-hermetic material, and the coating forms a hermetic seal over such elements to prevent air and water from entering the enclosed reservoir. In preferred embodiments, the DDD has an elongated, cylindrical shape and has a small enough outer diameter to permit in vivo insertion of the DDD using a narrow diameter applicator such as a syringe needle.

The materials and construction of the implantable DDDs described herein account for the competing constraints with respect to hermeticity, laser activation, implantability, and manufacturability of the devices. Some polymers are well suited to thin-walled construction and are compatible with laser activation. For example, some polymers may be easily manufactured as thin-walled elements and may be breached using a pulse of laser radiation. However, these polymers may not provide sufficiently low water vapor barrier characteristics required for some of the drugs of interest. Metals, glasses, and ceramics, on the other hand, offer superior barrier characteristics. These materials generally require higher laser energy to breach; however, the DDD described herein uses these materials in the form of thin walls and/or coatings, so that they can be breached with a minimal amount of laser energy.

In embodiments, the walls that form the enclosed reservoir are relatively thin (e.g., 1 μm to 100 μm), depending on the particular laser mechanism employed (thermal, thermo-mechanical, photo-chemical, photo-disruptive, etc.). In embodiments, the wall thickness ranges from 1 μm to 75 μm, preferably from 5 μm to 50 μm, and more preferably from 5 μm to 15 μm. If the DDD is to be injected into an ocular cavity, the enclosed reservoirs will typically have an internal cavity diameter ranging from 50 μm to 500 μm. Depending on the length of the DDD, the volume of the enclosed reservoir or reservoir section typically ranges from 0.1 μl to 10 μl. Larger values may be used depending on the method and site of implantation. The dimension range may be higher for non-ocular applications.

The drug units of the DDDs contain one or more drugs. The drug unit may contain one or more excipients. The drug unit may be in the form of an elongated tablet or a capsule. In a preferred embodiment, the drug unit is a microtablet formulated and made as described in U.S. Pat. No. 8,192,659 to Coppeta, et al., which is incorporated herein by reference.

The DDDs described herein can be used with essentially any drug, or active pharmaceutical ingredient (API). In a preferred embodiment, the drug is selected from potent biomolecules, such as proteins, antibodies, vaccines, RNA, DNA, or the like). In other embodiments, the drug is selected from small molecule pharmaceuticals. In one embodiment, the drug is an anti-VEGF drug. Examples of such drugs include the antibody fragment ranibizumab/Lucentis™, the antibody bevacizumab/Avastin™, and the fusion protein aflibercept/Eylea™. In other embodiments, the drug may be selected from the group consisting of anti-angiogenesis agents, anti-inflammatories, anti-infectives, anti-allergens, cholinergic agonists and antagonists, adrenergic agonists and antagonists, anti-glaucoma agents, agents for cataract prevention or treatment, neuroprotection agents, anti-oxidants, antihistamines, anti-platelet agents, anti-coagulants, anti-thrombic agents, anti-scarring agents, anti-proliferatives, anti-tumor agents, complement inhibitors, decongestants, vitamins, growth factors, anti-growth factor agents, gene therapy vectors, chemotherapy agents, protein kinase inhibitors, small interfering RNAs, antibodies, antibody fragments, fusion proteins, limus family compounds, and combinations thereof. Examples of suitable excipients include but are not limited to lyoprotectants, binding agents, buffers, surfactants, and/or slip agents, all of which are known in the art.

The following exemplary embodiments of implantable DDDs provide one or more hermetically-sealed reservoirs that are capable of providing a laser-activated release of a drug.

### A. Tube-and-End-Caps Device

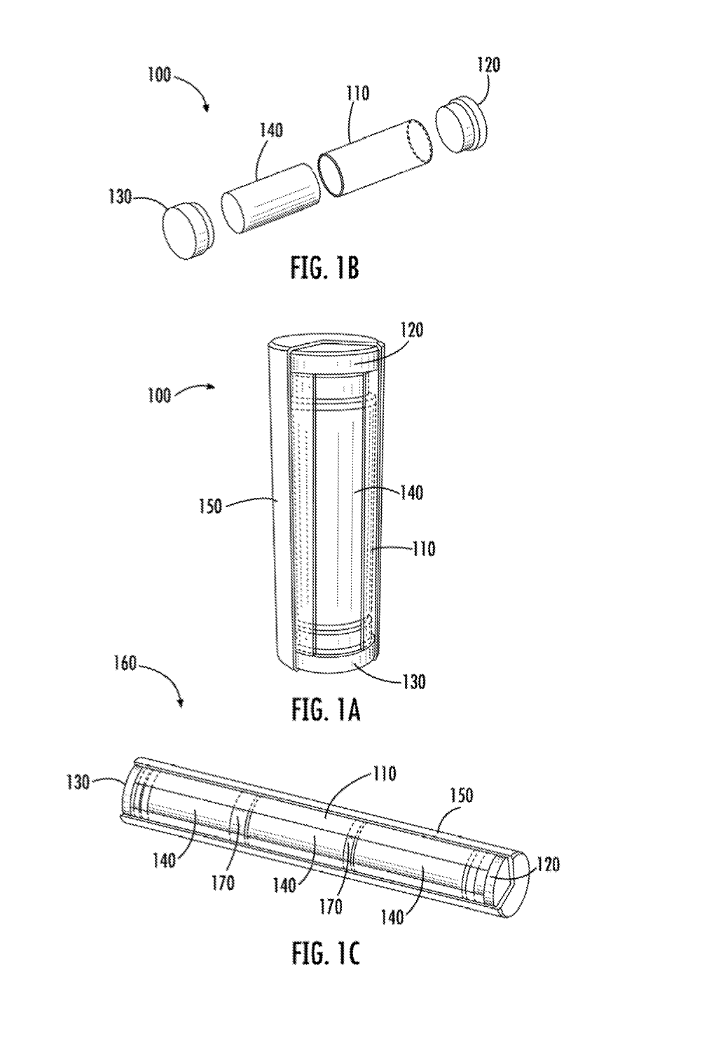

FIGS. 1A and 1B show an embodiment of an implantable DDD 100 including a tube element 110 having a first open end and a second open end. In some embodiments, the tube element 110 is formed of a glass, providing a highly impermeable barrier to air and water. The glass may be relatively brittle or fragile, and thus can be breached easily during laser activation of the DDD 100. Non-limiting examples of suitable glasses for the tube element 110 are semi-crystalline quartz, photo-lithographically constructed semiconductor structures, fused silica, and Apex photodefinable glass. In one embodiment, a thin-walled microcapillary glass tube is used as the tube element 110. A commercially-available example is part number TSP320450 produced by PolyMicro, Inc. This tube is a fused silica capillary having an outer diameter of 450 μm and an inner diameter of 320 μm, which corresponds to a glass wall thickness of 65 μm. Another example is part number BG-05 produced by Charles Supper Co., which has an outer diameter of 500 μm and a wall thickness of 10 to 15 μm. Thinner walls are desirable, such as walls having a thickness of 5 to 10 μm, but fabrication techniques may limit achievable wall thicknesses of microcapillary glass tubes. In one embodiment, the glass tube element 110 is able to absorb light irradiation from a laser source effective to breach the tube element 110. For example, the glass tube element 110 may be formed with an absorber. A commercially-available example of a glass formed with an absorber is RG1000 visible light absorbing glass produced by Schott Glass.

In some embodiments, the tube element 110 is formed of a ductile metal providing a highly impermeable barrier to air and water. Non-limiting examples of suitable metals for the tube element 110 are titanium and gold. In one embodiment, the metal tube element 110 is produced using conventional extrusion techniques or using a co-extrusion technique for ultra-thin walls (e.g., 5 to 10 μm). According to co-extrusion techniques, the core of a wire can be made of a selectively etchable material to create a hollow tube structure after etching. A commercially-available example of a co-extruded wire is produced by Anomet for the medical industry. In one embodiment, the metal tube element 110 is able to absorb light irradiation from a laser source effective to breach the tube element 110.

In other embodiments, the tube element 110 is formed of materials other than glass or metal, which materials are highly impermeable to air and water and are able to be breached easily during laser activation of the DDD 100.

The implantable DDD 100 also includes a first end cap element 120 and a second end cap element 130. The first end cap element 120 is joined to the first open end of the tube element 110 at a first joint, and the second end cap element 130 is joined to the second open end of the tube element 110 at a second joint. Accordingly, the tube element 110, the first end cap element 120, and the second end cap element 130 form an enclosed reservoir. In some embodiments, the end cap elements 120, 130 are formed of a metal providing a highly impermeable barrier to air and water. Non-limiting examples of suitable metals for the end cap elements 120, 130 are titanium and gold. In some embodiments, the end cap elements 120, 130 are formed of a glass, silicon, or other ceramic material. In one embodiment, as shown in FIG. 1A, the end cap elements 120, 130 each include a smaller diameter portion and a larger diameter portion. Accordingly, the end cap elements 120, 130 have a T-shaped cross-section and an axially symmetric shape. The smaller diameter portion is inserted into an open end of the tube element 110, and the larger diameter portion contacts an edge of the tube element 110, forming a joint. Accordingly, the end cap elements 120, 130 are partially received in the tube element 110 at the first joint and the second joint, respectively. In one embodiment, the end cap elements 120, 130 are formed as disks having a constant diameter, and the end cap elements 120, 130 are entirely received in the tube element 110 at the first joint and the second joint, respectively. In one embodiment, the end cap elements 120, 130 are formed as metal foils that are ultrasonically bonded to the open ends of the tube element 110.

In some embodiments, the integrity of the joints between the end cap elements 120, 130 and the tube element 110 is enhanced by use of an adhesive applied to the end cap elements 120, 130, the tube element 110, or both. In one embodiment, the adhesive is a polymer. Non-limiting examples of a suitable polymer include an epoxy, a thermoplastic polymer, a thermoset polymer, and other polymeric materials commonly used to create an adherent layer for bonding or sealing. In one embodiment, the adhesive is a pre-coating material. Non-limiting examples of suitable pre-coating materials are gold, titanium, platinum, and other pre-coating materials commonly used to create an adherent layer for bonding or sealing. In one embodiment, the adhesive is applied only to interfacing surfaces of the end cap elements 120, 130 and the tube element 110. In one embodiment, the adhesive is applied only to non-interfacing surfaces of the end cap elements 120, 130 and the tube element 110. In one embodiment, the adhesive is applied to interfacing surfaces and non-interfacing surfaces of the end cap elements 120, 130 and the tube element 110. Use of the adhesive is advantageous when the end cap elements 120, 130 and the tube element 110 are formed of dissimilar materials. For example, use of the adhesive is particularly advantageous when the end cap elements 120, 130 are formed of a metal and the tube element 110 is formed of a glass because the adhesive serves to bond and seal the joints of the dissimilar materials. Glass-metal seals of this type can be used in high vacuum applications in which pressures as low as 10⁻¹⁰ Torr are maintained, making this seal type an excellent choice for creating a hermetic seal between glass and metal elements. In one embodiment, the adhesive is applied to interfacing surfaces of the tube element 110 and/or the end cap elements 120, 130, and the end cap elements 120, 130 are inserted into (i.e., press-fit) or pressed onto the open ends of the tube element 110. In one embodiment, the end cap elements 120, 130 are inserted into or pressed onto the open ends of the tube element 110, and the adhesive is applied to non-interfacing surfaces of the tube element 110 and the end cap elements 120, 130.

In some embodiments, the integrity of the joints between the end cap elements 120, 130 and the tube element 110 is enhanced by welding or soldering the end cap elements 120, 130 to the tube element 110. The use of welding or soldering to bond the elements is particularly advantageous when the end cap elements 120, 130 and the tube element 110 are formed of a metal because the welding or soldering forms a hermetic seal over the first joint and the second joint, respectively. Non-limiting examples of welding or soldering techniques include ultrasonic welding, compression welding, resistive welding, cold-welding, and low-temperature soldering. In one embodiment, the end cap elements 120, 130 are coated with a metal that is amenable to cold welding, such as gold, titanium, or stainless steel, and the end cap elements 120, 130 are welded accordingly to the tube element 110. The coating material may be electroplated or vapor deposited onto the end cap elements 120, 130 to achieve the desired thickness. In one embodiment, the end cap elements 120, 130 are soldered to the tube element 110 with a biocompatible solder material that contains no silver or lead and is able to be used in medical grade devices.

In a variation of this embodiment, it is possible to omit any coatings from the DDD, as the welded tube and end cap elements themselves form the complete hermetic enclosure and provide a suitable laser-absorbing surface.

The implantable DDD 100 further includes at least one drug unit 140 contained in the enclosed reservoir formed by the tube element 110 and the end cap elements 120, 130. The drug unit 140 generally is inserted into the tube element 110 with one of the end cap elements 120, 130 already joined to the tube element 110. The other of the end cap elements 120, 130 is then joined to the tube element 110, enclosing the reservoir around the at least one drug unit 140. The drug unit 140 includes at least one drug.

In some embodiments, the implantable DDD 100 includes a coating 150 over all or a portion of the tube element 110 and the end cap elements 120, 130. In one embodiment, the coating 150 is formed of a metal providing a highly impermeable barrier to air and water. Non-limiting examples of suitable metals for the coating 150 include titanium and gold. In one embodiment, the coating 150 is formed of a glass, ceramic, metal alloy, metal laminate, or other hermetic material. In one embodiment, the coating 150 is less than 10 microns thick. In one embodiment, the coating 150 is formed of a titanium layer that is between 0.2 and 1 micron thick. In one embodiments, the first coating 150 is formed by physical deposition or another coating technique that produces a contiguous, highly impermeable and inert layer that is thermally coupled to the tube element 110 and the end cap elements 120, 130. Representative deposition techniques include sputtering, e-beam evaporative coating, plasma enhanced chemical vapor deposition, atomic layer deposition, and plasma enhanced chemical vapor deposition. In one embodiment, the coating 150 is formed over the joints of the end cap elements 120, 130 and the tube element 110. For example, when the end cap elements 120, 130 are joined to the tube element 110 by an adhesive, the coating 150 may be formed over the adhesive to provide a hermetic seal over the first joint and the second joint, respectively. In one embodiment, the coating 150 is formed over all of the end cap elements 120, 130. For example, when the end cap elements 120, 130 are formed of a non-hermetic material, the coating 150 may be formed over all of the end cap elements 120, 130 to provide a hermetic seal over the end cap elements 120, 130. In one embodiment, the coating 150 is formed over all of the tube element 110 and the end cap elements 120, 130. For example, when the tube element 110 and the end cap elements 120, 130 are formed of non-hermetic materials, the coating 150 may be formed over all of the tube element 110 and the end cap elements 120, 130 to provide a hermetic seal over the tube element 110 and the end cap elements 120, 130. In one embodiment, the coating 150 is formed over all or a portion of the tube element 110, and the coating 150 is able to absorb light irradiation. For example, when the tube element 110 is formed of a non-irradiation-absorbing material, the coating 150 may be formed over all or a portion of the tube element 110 to absorb light irradiation effective to breach the tube element 110 to permit release of the drug. In one embodiment, the DDD 100 includes more than one coating 150. For example, the DDD 100 may include one coating 150 formed over one or more elements to provide a hermetic seal and another coating 150 formed over the tube element 110 to absorb light irradiation effective to breach the tube element 110 to permit release of the drug.

In some embodiments, a plurality of DDDs 100 are joined together to provide a single device having multiple enclosed reservoirs. Accordingly, the plurality of DDDs 100 may be implanted in a tissue together but activated separately by multiple laser activation events. In one embodiment, the DDDs 100 are joined by a flexible tether. In one embodiment, the DDDs 100 are disposed within a polymeric sheath, which serves primarily to hold the DDDs together, typically in an axial, or end-to-end arrangement.

In another embodiment, the tube-and-end-caps device of FIGS. 1A and 1B is modified to provide a single device having multiple reservoir sections. FIG. 1C depicts an embodiment of an implantable DDD 160 including a tube element 110, a first end cap element 120, a second end cap element 130, and a coating 150. The tube element 110, the first end cap element 120, and the second end cap element 130 form an enclosed reservoir. The DDD 160 also includes at least one barrier element 170 positioned within the enclosed reservoir and defining a plurality of separate reservoir sections. The DDD 160 further includes a plurality of drug units 140 distributed within the separate reservoir sections. In one embodiment, the at least one barrier element 170 includes a plurality of barrier elements 170 positioned within the enclosed reservoir and defining a plurality of separate reservoir sections. In one embodiment, the plurality of drug units 140 is distributed such that each of the separate reservoir sections contains one drug unit 140. In one embodiment, the plurality of drug units 140 is distributed such that one or more of the separate reservoir sections contains multiple drug units 140. In one embodiment, the at least one barrier element 170 and the plurality of drug units 140 are arranged such that multiple drug doses may be released sequentially (and in spaced intervals) from the DDD 160 using a single laser activation event. This single-activation-multiple-releases embodiment is highly advantageous from a patient and physician perspective.

In some embodiments, the at least one barrier element 170 is formed of an erodible polymer. Accordingly, the at least one barrier element 170 allows for a time-delayed release of multiple doses from a single laser activation event. For example, one of the reservoir sections may be breached by a laser activation event to permit release of a first drug, and subsequent erosion of the at least one barrier element 170 from exposure to a fluid permits release of a second drug from an adjacent reservoir section. In one embodiment, the at least one barrier element 170 is formed of a polymer from the class of polyanhydrides or other polymers that are relatively hydrophobic and that degrade over time by a surface erosion mechanism. Accordingly, the at least one barrier element 170 permits release of a drug from a reservoir section after exposure to a fluid for a pre-determined period of time. In one embodiment, the at least one barrier element 170 is formed of a relatively hydrophilic “bulk-eroding” polymer that is highly permeable or soluble. Accordingly, the at least one barrier element 170 permits release of a drug from a reservoir section soon after exposure to a fluid. In one embodiment, the at least one barrier element 170 includes a plurality of barrier elements 170, at least one of which is formed of a hydrophobic polymer and at least one of which is formed of a hydrophilic polymer to create a custom and complex drug release profile.

In a further embodiment, the tube-and-end-caps design of FIGS. 1A and 1B is modified to provide a single device having multiple enclosed reservoirs. FIG. 1D depicts an embodiment of an implantable DDD 180 including a plurality of tube elements 110, a first end cap element 120, a second end cap element 130, and a coating 150. The DDD 180 also includes at least one intermediate cap element 190 positioned between and joined to adjacent tube elements 110. Accordingly, the plurality of tube elements 110, the first end cap element 120, the second end cap element 130, and the at least one intermediate cap element 190 form a plurality of enclosed reservoirs. In some embodiments, the intermediate cap element 190 is formed of a metal providing a highly impermeable barrier to air and water. Non-limiting examples of suitable metals for the intermediate cap element 190 are titanium and gold. In some embodiments, the intermediate cap element 190 is formed of a glass, silicon, or other ceramic material. In one embodiment, the intermediate cap element 190 includes two smaller diameter portions separated by a larger diameter portion. One of the smaller diameter portions is inserted into an open end of one adjacent tube element 110, and the larger diameter portion contacts an edge of the one adjacent tube element 110, forming a joint. The other smaller diameter portion is inserted into an open end of the other adjacent tube element 110, and the larger diameter portion contacts an edge of the other adjacent tube element 110, forming another joint. Accordingly, the intermediate cap element 190 allows for end-to-end stacking of adjacent tube elements 110. The DDD 180 further includes a plurality of drug units 140 distributed within the enclosed reservoirs. In one embodiment, the plurality of drug units 140 is distributed such that each of the enclosed reservoirs contains one drug unit 140. In one embodiment, the plurality of drug units 140 is distributed such that one or more of the enclosed reservoirs contains multiple drug units 140. In some embodiments, the at least one intermediate cap element 190 and the plurality of drug units 140 are arranged such that the plurality of drug units 140 may be selectively released from the DDD 160 by multiple laser activation events. For example, one of the reservoirs may be breached by a first laser activation event to permit release of a first drug, while an adjacent reservoir remains intact for activation at a later point in time.

### B. Spaced-Cups Device

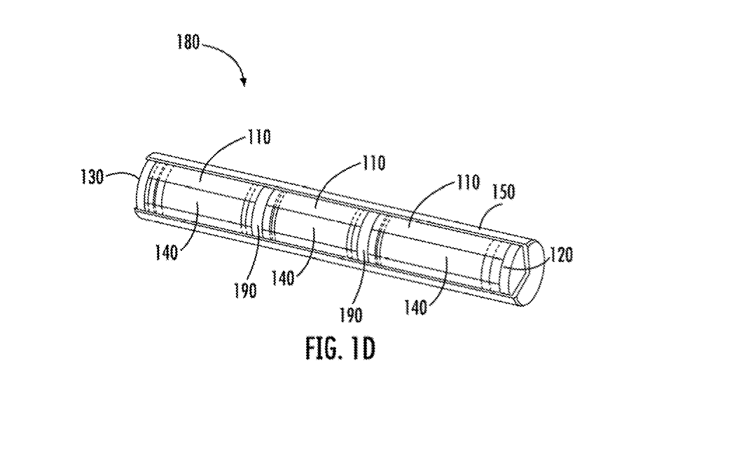

FIGS. 2A and 2B depict another embodiment of an implantable DDD 200 including a first cup element 210 and a second cup element 220. The first cup element 210 has an open end and a closed end, and the second cup element 220 similarly has an open end and a closed end. In some embodiments, the cup elements 210, 220 are formed of a metal providing a highly impermeable barrier to air and water. Non-limiting examples of suitable metals for the cup elements 210, 220 are titanium and gold. The cup elements 210, 220 are oriented such that the open end of the first cup element 210 faces the open end of the second cup element 220. Further, the cup elements 210, 220 are spaced apart from one another to define a gap between the open end of the first cup element 210 and the open end of the second cup element 220.

The DDD 200 also includes a coating 230 over all or a portion of the cup elements 210, 220 such that the coating 230 fills the gap and forms a hermetic seal between the cup elements 210, 220. Accordingly, the cup elements 210, 220 and the coating 230 form an enclosed reservoir. In some embodiments, the coating 230 is formed of a metal providing a highly impermeable barrier to air and water. Non-limiting examples of suitable metals for the coating 230 are titanium and gold. In some embodiments, the coating 230 is formed of an epoxy, ceramic, or atomic layer deposited (ALD) material. In some embodiments, the coating 230 is able to absorb light irradiation from a laser source effective to open the enclosed reservoir. In some embodiments, the coating 230 covers all of the cup elements 210, 220, forming a hermetic seal over the entire DDD 200.

The DDD 200 further includes at least one drug unit 240 contained in the enclosed reservoir formed by the cup elements 210, 220 and the coating 230. The at least one drug unit 240 includes a drug. In some embodiments, the at least one drug unit 240 also includes an excipient. In some embodiments, the drug unit 240 is formed as an elongated tablet or microtablet.

The DDD 200 also includes a tube element 250 having a first open end and a second open end. The tube element 250 is contained in the enclosed reservoir and is positioned over at least a portion of the drug unit 240. Further, the tube element 250 is positioned between the drug unit 240 and the coating 230. Specifically, the tube element 250 is positioned between the drug unit 240 and the portion of the coating 230 that fills the gap between the cup elements 210, 220. Accordingly, the tube element 250 protects the drug unit 240 during assembly of the DDD 200, particularly during application of the coating 230. The first cup element 210 overlaps the first open end of the tube element 250, and the second cup element 220 overlaps the second open end of the tube element 250. In some embodiments, the tube element 250 has a length greater than the combined lengths of the cup elements 210, 220. Accordingly, the tube element 250 assists in creating the gap between the cup elements 210, 220 during assembly of the DDD 200. In some embodiments, the tube element 250 is formed of a polymer providing a highly impermeable barrier to the coating 230 material. Non-limiting examples of suitable materials for the tube element 250 include polyester, polyurethane, and silicone.

### C. Overlapping-Cups Device

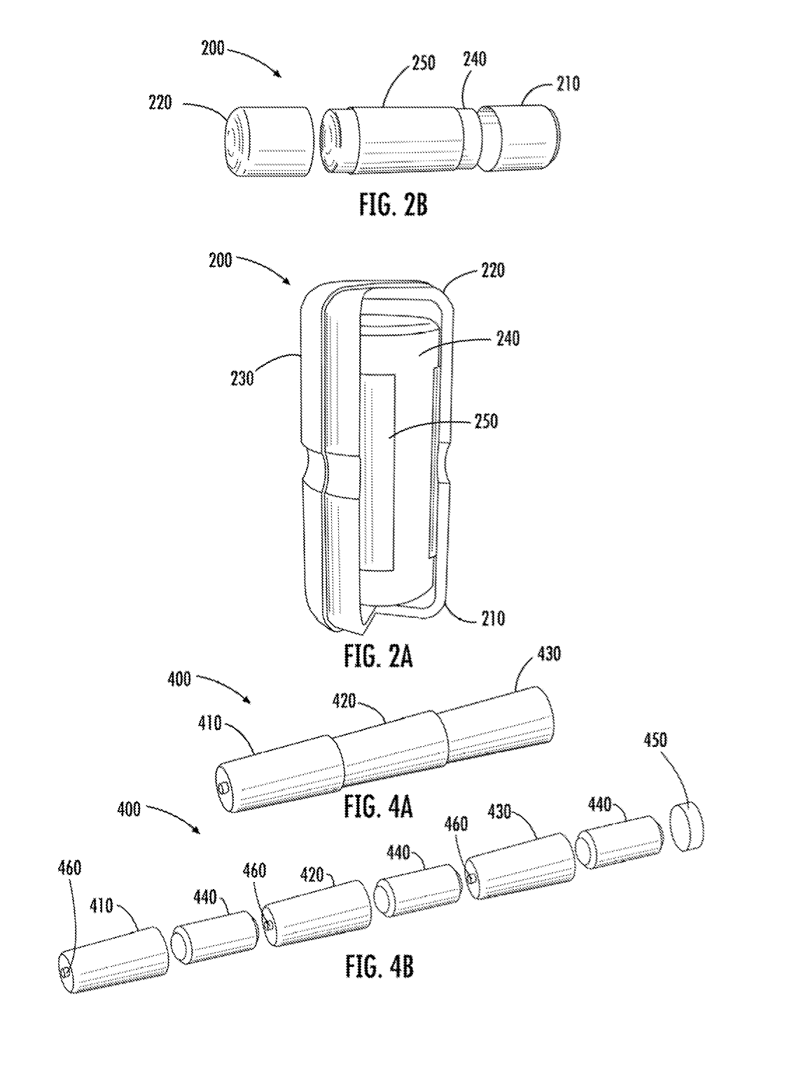

FIGS. 3A and 3B depict another embodiment of an implantable DDD 300 including a first cup element 310 and a second cup element 320. The first cup element 310 has an open end and a closed end, and the second cup element 320 similarly has an open end and a closed end. The cup elements 310, 320 are positioned such that the open end of the first cup element 310 overlaps the open end of the second cup element 320, forming a joint. In some embodiments, the cup elements 310, 320 are formed of a polymer. Non-limiting examples of suitable polymers for the cup elements 310, 320 are poly(p-xylylene) polymer (e.g., Parylene®), polyethylene, or another polyolefin that can be doped easily with an irradiation-absorbing material and can be locally melted or ablated easily by light irradiation.

The DDD 300 also includes a coating 330 over all or a portion of the cup elements 310, 320 such that the coating 330 forms a hermetic seal over the joint of the cup elements 310, 320. Accordingly, the cup elements 310, 320 and the coating 330 form an enclosed reservoir. In some embodiments, the coating 330 is formed of a metal providing a highly impermeable barrier to air and water. Non-limiting examples of suitable metals for the coating 330 are titanium and gold. In some embodiments, the coating 330 is formed of an epoxy, ceramic, or atomic layer deposited (ALD) material. In some embodiments, the coating 330 is able to absorb light irradiation from a laser source effective to open the enclosed reservoir. In some embodiments, the coating 330 covers all of the cup elements 310, 320, forming a hermetic seal over the entire DDD 300.

The DDD 300 further includes at least one drug unit 340 contained in the enclosed reservoir formed by the cup elements 310, 320 and the coating 330. The at least one drug unit 340 includes a drug. In some embodiments, the at least one drug unit 340 also includes an excipient. In some embodiments, the drug unit 340 is formed as an elongated tablet or microtablet.

### D. Stackable-Cups Device

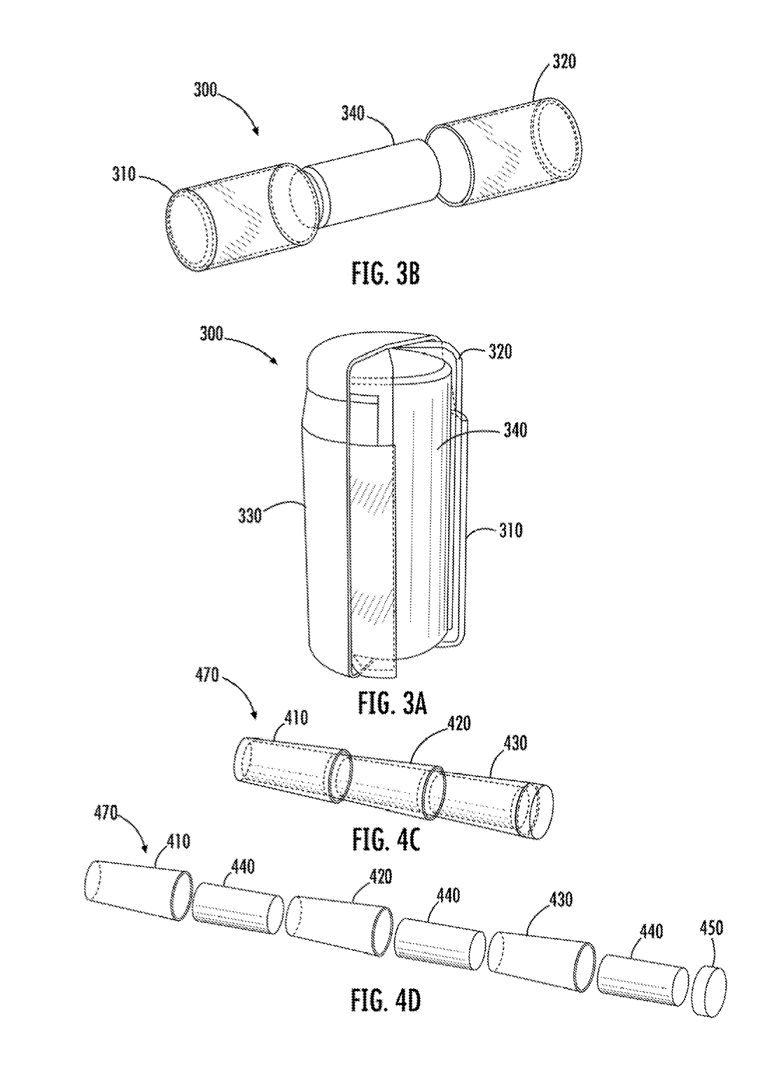

FIGS. 4A and 4B depict another embodiment of an implantable DDD 400 including a first cup element 410, a second cup element 420, and a third cup element 430. The cup elements 410, 420, 430 each have an open end and a closed end. The first and second cup elements 410, 420 are positioned such that the open end of the first cup element 410 overlaps the closed end of the second cup element 420, forming a joint. The second and third cup elements 420, 430 are positioned such that the open end of the second cup element 410 overlaps the closed end of the third cup element 420, forming another joint. Accordingly, open and closed ends of the cup elements 410, 420, 430 allow for end-to-end stacking. The inner cavity of the first cup element 410 and the closed end of the second cup element 420 form an enclosed reservoir. Further, the inner cavity of the second cup element 420 and the closed end of the third cup element 430 form another enclosed reservoir. Accordingly, the DDD 400 provides a single device having a plurality of enclosed reservoirs. In some embodiments, the cup elements 410, 420, 430 are formed of a metal providing a highly impermeable barrier to air and water. Non-limiting examples of suitable metals for the cup elements 410, 420, 430 are titanium and gold. In some embodiments, one or more of the cup elements 410, 420, 430 are able to absorb light irradiation from a laser source effective to open one of the enclosed reservoirs. In some embodiments, one or more of the cup elements 410, 420, 430 are able to absorb light irradiation from a laser source effective to open all of the enclosed reservoirs. In some embodiments, the DDD 400 includes additional cup elements forming additional enclosed reservoirs.

The DDD 400 also includes a plurality of drug units 440 distributed within the plurality of enclosed reservoirs formed by the cup elements 410, 420, 430. The drug unit 440 includes a drug. In some embodiments, the drug unit 440 also includes an excipient. In some embodiments, the drug unit 440 is formed as an elongated tablet or microtablet. In one embodiment, the plurality of drug units 440 is distributed such that each of the enclosed reservoirs contains one drug unit 440. In one embodiment, the plurality of drug units 440 is distributed such that one or more of the enclosed reservoirs contains multiple drug units 440. In some embodiments, the cup elements 410, 420, 430 and the plurality of drug units 440 are arranged such that the plurality of drug units 440 may be released from the DDD 400 by a single laser activation event. In some embodiments, the cup elements 410, 420, 430 and the plurality of drug units 440 are arranged such that the plurality of drug units 440 may be selectively released from the DDD 400 by multiple laser activation events. In some embodiments, the cup elements 410, 420, 430 are positioned within a polymer sleeve or other envelope structure.

In some embodiments, the DDD 400 includes an end cap element 450 joined to the open end of the third cup element 430 or other outermost cup element. Accordingly, the end cap element 450 and the inner cavity of the cup element form another enclosed reservoir for containing a drug unit 440. In some embodiments, the end cap element 450 is formed of a metal providing a highly impermeable barrier to air and water. Non-limiting examples of suitable metals for the end cap element 450 are titanium and gold. In some embodiments, the end cap element 450 is formed of a glass, silicon, or other ceramic material. In one embodiment, the end cap element 450 is a metal foil that is ultrasonically bonded to the open end of the cup element 430. In some embodiments, the end cap element 450 is able to absorb light irradiation from a laser source effective to open the enclosed reservoir to permit release of the drug unit 440.

In some embodiments, the DDD 400 includes one or more erodible plugs 460 positioned between adjacent enclosed reservoirs. The plugs 460 are able to erode over time and open a conduit between the adjacent reservoirs. Accordingly, an erodible plug 460 is able to permit release of a drug unit 440 from one adjacent reservoir once the other adjacent reservoir is empty. In this manner, the DDD 400 is arranged such that the plurality of drug units 440 may be released from the DDD 400 by a single laser activation event.

In some embodiments, the DDD 400 includes a coating (not shown) over all or a portion of the cup elements 410, 420, 430 such that the coating forms a hermetic seal over the joints between the cup elements 410, 420, 430. In some embodiments, the coating is formed of a metal providing a highly impermeable barrier to air and water. Non-limiting examples of suitable metals for the coating are titanium and gold. In some embodiments, the first coating is formed of a glass, ceramic, metal alloy, metal laminate, or other hermetic material. In some embodiments, the coating is able to absorb light irradiation from a laser source effective to open one of the enclosed reservoirs to permit release of one of the drug units 440. In some embodiments, the coating covers all of the cup elements 410, 420, 430, forming a hermetic seal over the entire DDD 400.

FIGS. 4C and 4D depict another embodiment of an implantable DDD 470 including a first cup element 410, a second cup element 420, a third cup element 430, a plurality of drug units 440, and an end cap element 450. The cup elements 410, 420, 430 are stacked end to end, forming a plurality of enclosed reservoirs. In some embodiments, the cup elements 410, 420, 430 are formed of a polymer. Non-limiting examples of suitable polymers for the cup elements 410, 420, 430 are polyp-xylylene) polymer (e.g., Parylene®), polyethylene, or another polyolefin that can be doped easily with an irradiation-absorbing material and can be locally melted or ablated easily by light irradiation. In some embodiments, the DDD 470 also includes one or more intermediate cap elements 480 positioned between adjacent enclosed reservoirs. In one embodiment, the intermediate cap elements are formed of a metal providing a highly impermeable barrier to air and water. Non-limiting examples of suitable metals for the intermediate cap elements 480 are titanium and gold. In one embodiment, the intermediate cap element 480 is formed as a disk and inserted into an enclosed reservoir before or after inserting a drug unit 440 into the enclosed reservoir. In some embodiments, the cup elements 410, 420, 430 and the plurality of drug units 440 are arranged such that the plurality of drug units 440 may be released from the DDD 470 by a single laser activation event. In some embodiments, the intermediate cap elements 480 and the plurality of drug units 440 are arranged such that the plurality of drug units 440 may be selectively released from the DDD 470 by multiple laser activation events. In some embodiments, the cup elements 410, 420, 430 are positioned within a polymer sleeve or other envelope structure.

In some embodiments, the DDD 470 includes a coating (not shown) over all or a portion of the cup elements 410, 420, 430 such that the coating forms a hermetic seal over the joints between the cup elements 410, 420, 430. In some embodiments, the coating is formed of a metal providing a highly impermeable barrier to air and water. Non-limiting examples of suitable metals for the coating are titanium and gold. In some embodiments, the first coating is formed of a glass, ceramic, metal alloy, metal laminate, or other hermetic material. In some embodiments, the coating is able to absorb light irradiation from a laser source effective to open one of the enclosed reservoirs to permit release of one of the drug units 440. In some embodiments, the coating covers all of the cup elements 410, 420, 430, forming a hermetic seal over the entire DDD 400. In some embodiments

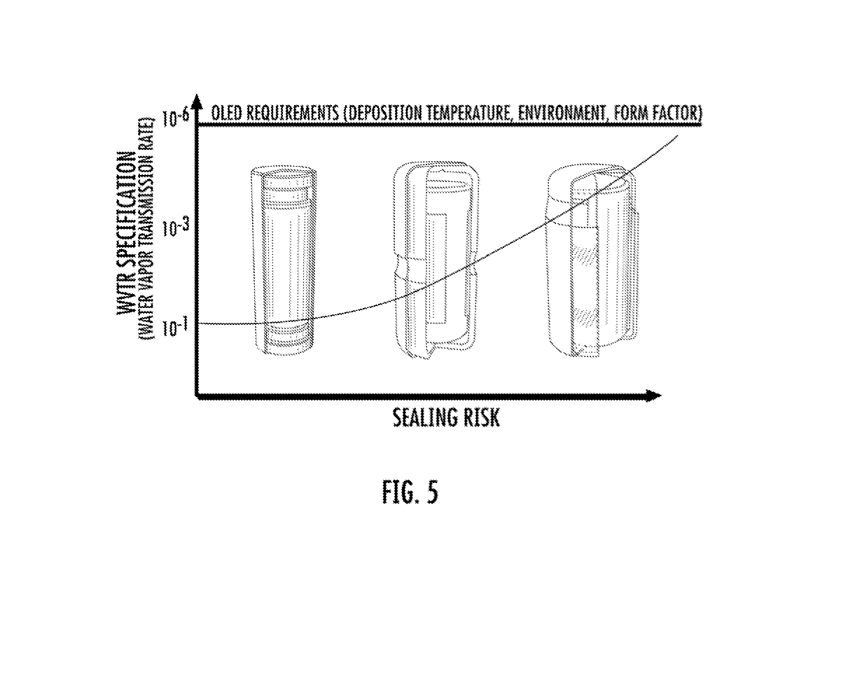

As illustrated in FIG. 5, each of the foregoing implantable DDD embodiments has unique advantages when taking into consideration the design constraints for water vapor transmission rate, mechanical or structural integrity, simplicity and ease of manufacture and sealing, and ease of laser activation. For example, embodiments of the tube-and-end-caps device (FIGS. 1A-1D) including a glass tube element and a metal coating advantageously provide a mechanically robust structure with a large laser activation targeting area capable and have a relatively low risk of a hermetic seal failure. Alternatively, embodiments of the spaced-cup device (FIGS. 2A and 2B) are highly volume efficient, have significant flexibility in material choice, and have a moderate risk of a hermetic seal failure. Lastly, embodiments of the overlapping-cups device (FIGS. 3A and 3B) including polymer cup elements are highly volume efficient and enable direct laser activation using lower power lasers. Accordingly, those skilled in the art should be able to select appropriate embodiments and materials depending on the desired balance of considerations.

For example, those skilled in the art evaluating embodiments illustrated in FIG. 4A and FIG. 4C will appreciate that the primary difference between the devices of FIG. 4A and FIG. 4C is the selection of the materials of construction. The cup elements 410, 420, 430 of implantable DDD 400 of FIG. 4A are constructed primarily of a hermetic material, such as a metal, whereas the cup elements 410, 420, 430 of implantable DDD 470 of FIG. 4C are constructed primarily of a non-hermetic polymer. Accordingly, the cup elements 410, 420, 430 of DDD 470 are coated with a hermetic material to provide a hermetic seal over the enclosed reservoirs. Not wishing to be bound by any theory, it is believed that DDD 400 would be easier to hermetically seal because only the joints between adjacent cup elements would require a hermetic coating. Conversely, DDD 470 would be more difficult to hermetically seal because a high quality hermetic coating would be required over nearly all of the outer surface of the device. Although it is believed that DDD 400 would be easier to hermetically seal than DDD 470, those skilled in the art would appreciate that DDD 400 likely would be more difficult to activate.

### 2. Laser Activation

The laser-activated DDDs described herein facilitate non-invasive release of a drug in the ocular tissue being treated, such as the macula or retina. For example, the DDDs may permit release of the drug into the posterior chamber through the vitreous portion of the eye or through the conjunctiva, sclera, or choroid. In addition to being non-invasive, laser activation allows for multiple dosing from a single DDD having multiple reservoirs or reservoir sections.

The DDDs permit release a drug payload when triggered by a pulse of light irradiation. In some embodiments, the light irradiation source is a focused laser. In some embodiments, as described above, the DDD includes a structural element and/or a coating formed of an irradiation-absorbing material. Accordingly, the structural element and/or coating is able to absorb the pulse of light irradiation, which heats the structural element and/or coating as well as adjacent elements of the DDD. In some embodiments, the structural element and/or coating is formed of an irradiation-absorbing material having a high optical absorption coefficient at a wavelength appropriate to a laser device to be controlled by a user for release of the chemical substance. In some embodiments, as described above, the structural element and/or coating has a wall thickness that is substantially thinner and more mechanically fragile than other elements of the DDD. Accordingly, a breach may be formed in the structural element and/or coating upon sufficient heating from the light irradiation. For example, the structural element and/or coating may fracture or melt near the area where the light irradiation is applied. In some embodiments the heating of the structural element and/or coating is sufficient to form a breach in an adjacent element surrounding the enclosed reservoir.

In some embodiments, the tube-and-end-caps device 100 of FIGS. 1A and 1B includes a tube element 110 having a thin wall thickness and formed of a material that is able to absorb the light irradiation. Accordingly, the tube element 110 may fracture upon application of the light irradiation, opening the enclosed reservoir to permit release of the drug. In some embodiments, the tube-and-end-caps device 100 includes a thin coating 150 formed over all or a portion of the tube element 110 and thermally coupled to the tube element 110. The coating 150 may be formed of an irradiation-absorbing material, and the tube element 110 may be formed of a material that does not absorb light irradiation. Accordingly, the coating 150 and the tube element 110 may fracture upon application of the light irradiation, opening the enclosed reservoir to permit release of the drug. In some embodiments, the tube-and-end-caps devices 160 and 180 of FIGS. 1C and 1D similarly include a tube element 110 or a coating 150 that is able to absorb light irradiation effective to open the enclosed reservoir to permit release of the drug.

In some embodiments, the spaced-cups device 200 of FIGS. 2A and 2B includes a coating 230 having a thin wall thickness and formed of a material that is able to absorb the light irradiation. Accordingly, the coating 230 may fracture upon application of the light irradiation, opening the enclosed reservoir to permit release of the drug. Specifically, the coating 230 may fracture in the portion of the coating 230 that fills the gap between the cup elements 210, 220. In some embodiments, the cup elements 210, 220 have a thin wall thickness and are formed of a material that is able to absorb the light irradiation. Accordingly, one or both of the cup elements 210, 220 may fracture upon application of the light irradiation, opening the enclosed reservoir to permit release of the drug.

In some embodiments, the overlapping-cups device 300 of FIGS. 3A and 3B includes a thin coating 330 formed over all or a portion of the cup elements 310, 320 and thermally coupled to the cup elements 310, 320. The coating 330 may be formed of an irradiation-absorbing material, and the cup elements 310, 320 may be formed of a material that does not absorb light irradiation. Accordingly, the coating 330 and the cup elements 310, 320 may fracture upon application of the light irradiation, opening the enclosed reservoir to permit release of the drug.

In some embodiments, the cup elements 310, 320 have a thin wall thickness and are formed of a material that is able to absorb the light irradiation. Accordingly, one or both of the cup elements 310, 320 may fracture upon application of the light irradiation, opening the enclosed reservoir to permit release of the drug.

In some embodiments, the stackable-cups devices 400, 470 of FIGS. 4A-4D include cup elements 410, 420, 430 having a thin wall thickness and formed of a material that is able to absorb the light irradiation. Accordingly, one of the cup elements 410, 420, 430 may fracture upon application of the light irradiation, opening one of the enclosed reservoirs to permit release of the drug. In some embodiments, the stackable-cups devices 400, 470 include a thin coating formed over all or a portion of the cup elements 410, 420, 430 and thermally coupled to the cup elements 410, 420, 430. The coating may be formed of an irradiation-absorbing material, and the cup elements 410, 420, 430 may be formed of a material that does not absorb light irradiation. Accordingly, the coating and one of the cup elements 410, 420, 430 may fracture upon application of the light irradiation, opening one of the enclosed reservoirs to permit release of the drug.

### 3. Methods of Using the Drug Delivery Devices

The implantable drug delivery devices described herein may be used to deliver a drug to a patient. Particularly, the implantable devices facilitate selective release of a drug to the interior of the eye for the treatment of ocular conditions. However, the implantable devices may be adapted for use in other parts of the body.

One embodiment includes a method of delivering a drug to a patient by use of a laser-activated drug-delivery device. The method includes implanting one of the above-described drug delivery devices into a tissue site of the patient. The device includes a drug contained in an enclosed reservoir. The method also includes irradiating at least a portion of the drug delivery device to breach the enclosed reservoir to permit the drug to be released in tissues at the tissue site.

In some embodiments, the tissue site is ocular tissue. In one embodiment, the tissue site is in the posterior chamber of the eye. In one embodiment, the tissue site is in, on, or under the conjunctiva of the eye. In one embodiment, the tissue site is in, on, or under the sclera of the eye. In one embodiment, the tissue site is in, on, or under the choroid of the eye.

Publications cited herein and the materials for which they are cited are specifically incorporated by reference. Modifications and variations of the methods and devices described herein will be obvious to those skilled in the art from the foregoing detailed description. Such modifications and variations are intended to come within the scope of the appended claims

We claim:

1. An implantable drug delivery device comprising:
a tube element having a first open end and a second open end;
a first end cap element joined to the first open end of the tube element at a first joint, wherein the first joint includes interfacing surfaces of the first end cap element and the tube element;
a second end cap element joined to the second open end of the tube element at a second joint, wherein the second joint includes interfacing surfaces of the second end cap element and the tube element; and wherein the tube element, the first end cap element, and the second end cap element form an enclosed reservoir;
at least one drug unit contained in the enclosed reservoir, wherein the at least one drug unit comprises a drug; and
a coating on at least a portion of non-interfacing surfaces of the tube element, the first end cap element, and the second end cap element, wherein the coating forms a hermetic seal of the first joint and the second joint;
wherein the implantable drug delivery device is configured to absorb light irradiation from a laser source effective to open the enclosed reservoir and thereby permit release of the drug.

2. The drug delivery device of claim 1, wherein the first end cap element is joined to the first open end of the tube element by an adhesive at the first joint, and wherein the second end cap element is joined to the second open end of the tube element by an adhesive at the second joint.

3. The drug delivery device of claim 2, wherein the adhesive is applied to interfacing surfaces of the tube element, the first end cap element, and the second end cap element.

4. The drug delivery device of claim 2, wherein the adhesive is applied to non-interfacing surfaces of the tube element, the first end cap element, and the second end cap element.

5. The drug delivery device of claim 2, wherein the adhesive comprises an epoxy.

6. The drug delivery device of claim 2, wherein the coating forms a hermetic seal over the adhesive at the first joint and the second joint.

7. The drug delivery device of claim 1, wherein the tube element is configured to absorb the light irradiation.

8. The drug delivery device of claim 1, wherein the tube element is formed of a metal.

9. The drug delivery device of claim 8, wherein the metal comprises titanium, gold, or a combination thereof.

10. The drug delivery device of claim 1, wherein the tube element has a wall thickness between 5 μm and 75 μm.

11. The drug delivery device of claim 1, wherein the first end cap element and the second end cap element are formed of a metal.

12. The drug delivery device of claim 1, wherein the tube element is formed of a glass.

13. The drug delivery device of claim 1, wherein the coating is configured to absorb the light irradiation.

14. The drug delivery device of claim 1, wherein the coating comprises a metal.

15. The drug delivery device of claim 14, wherein the metal of the coating comprises titanium, platinum, gold, or a combination thereof.

16. The drug delivery device of claim 1, wherein the coating has a thickness of less than 1 μm.

17. The drug delivery device of claim 1, wherein the first end cap element and the second end cap element are formed of a non-hermetic material, and wherein the coating forms a hermetic seal over the first end cap element and the second end cap element.

18. The drug delivery device of claim 1, wherein the tube element, the first end cap element, and the second end cap element are formed of non-hermetic materials, and wherein the coating forms a hermetic seal over all of the tube element, the first end cap element, and the second end cap element.

19. The drug delivery device of claim 1, wherein the first end cap element is partially received in the tube element at the first joint, and wherein the second end cap element is partially received in the tube element at the second joint.

20. The drug delivery device of claim 1, wherein the first end cap element comprises a smaller diameter portion and a larger diameter portion, wherein the second end cap element comprises a smaller diameter portion and a larger diameter portion, wherein the smaller diameter portion of the first end cap element is received in the tube element at the first joint, and wherein the smaller diameter portion of the second end cap element is received in the tube element at the second joint.

21. The drug delivery device of claim 1, wherein the at least one drug unit comprises an elongated tablet.

22. The drug delivery device of claim 1, wherein the drug comprises a protein, antibody, antibody fragment, vaccine, RNA, or DNA.

23. The drug delivery device of claim 1, wherein the drug comprises ranibizumab, bevacizumab, aflibercept, or another anti-VEGF drug.

24. The drug delivery device of claim 1, further comprising a plurality of barrier elements positioned within the enclosed reservoir and defining a plurality of separate reservoir sections, wherein the at least one drug unit comprises a plurality of drug units distributed within the separate reservoir sections.

25. A device comprising two or more of the drug delivery devices of claim 1 secured together to form a single device.

26. The device of claim 25, wherein the two or more devices are disposed within a sheath in an axial or end-to-end arrangement.

27. The device of claim 25, wherein:
each of the two or more devices includes a plurality of barrier elements positioned within the enclosed reservoir and defining a plurality of separate reservoir sections, and
the at least one drug unit comprises a plurality of drug units distributed within the separate reservoir sections.

28. The device of claim 27, wherein the barrier elements are erodible in vivo, so that the device is operable to provide time-delayed release of multiple doses of the drug from a single laser activation event.

29. An implantable drug delivery device comprising:
a first cup element having an open end and a closed end;
a second cup element having an open end and a closed end;
a coating over all or a portion of the first cup element and the second cup element, wherein the coating forms a hermetic seal between the first cup element and the second cup element, and wherein the first cup element, the second cup element, and the coating form an enclosed reservoir; and
at least one drug unit contained in the enclosed reservoir, wherein the at least one drug unit comprises a drug,
wherein the implantable drug delivery device is configured to absorb light irradiation from a laser source effective to open the enclosed reservoir to permit release of the drug, and
wherein:
the first cup element and the second cup element are spaced apart from one another to define a gap, and wherein the coating fills the gap, or
the open end of the first cup element overlaps the open end of the second cup element.

30. The drug delivery device of claim 29, wherein the coating is configured to absorb the light irradiation.

31. The drug delivery device of claim 29, wherein the coating is formed of a metal.

32. A method of delivering a drug to a patient, comprising:
implanting the drug delivery device of claim 1 into a tissue site of the patient, wherein the drug delivery device comprises a drug contained in a hermetically enclosed reservoir; and
irradiating at least a portion of the drug delivery device with laser energy in an amount effective to breach the enclosed reservoir to permit the drug to be released in tissues at the tissue site.

33. The method of claim 32, wherein the tissue site comprises ocular tissue.
